SEQUENCE LISTING (22 sequences)

SEQ ID NO: 1 - protein, 62 aa, Naja atra or Naja kaouthia
LECHNQQSSQTPTTTGCSGGETNCYKKRWRDHRGYRTERGCGCPSVKNGIEINCCTTDRCNN

SEQ ID NO: 2 - protein, 83 aa, Naja atra or Naja kaouthia
MKTLLLTLLVVTIVCLDLGYTLECHNQQSSQTPTTTGCSGGETNCYKKRWRDHRGYRTERGCGCPSVKNGIEINCCTTDRCNN

SEQ ID NO: 3 - protein, 62 aa, Naja atra or Naja kaouthia
LECHNQQSSQTPTTTGCSGGETNCYKKRWRDHRGYRTERGCGCPIVKNGIESNCCTTDRCNN

SEQ ID NO: 4 - protein, 61 aa, Naja atra or Naja kaouthia
MECHNQQSSQAPTTKTCSGETNCYKKWWSDHRGTIIERGCGCPKVKPGVNLNCCTTDRCNN

SEQ ID NO: 5 - protein, 62 aa, Naja atra or Naja kaouthia
MECHNQQSSQTPTTTGCSGGETNCYKKWWSDHRGTIIERGCGCPKVKPGVNLNCCTTDRCNN

SEQ ID NO: 6 - protein, 61 aa, Naja atra or Naja kaouthia
LECHNQQSSQTPTTKTCSGETNCYKKWWSDHRGTIIERGCGCPKVKPGVNLNCCTTDRCNN

SEQ ID NO: 7 - protein, 74 aa, Bungarus multicinctus
IVCHTTATSPISAVTCPPGENLCYRKMWCDAFCSSRGKVVELGCAATCPSKKPYEEVTCCSTDKCNPHPKQRPG

SEQ ID NO: 8 - protein, 95 aa, Bungarus multicinctus
MKTLLLTLVVVTIVCLDLGYTIVCHTTATSPISAVTCPPGENLCYRKMWCDAFCSSRGKVVELGCAATCPSKKPYEEVTCCSTDKCNPHPKQRPG

SEQ ID NO: 9 - protein, 60 aa, Dendroaspis polylepis
  misc_feature (1)..(1): Xaa can be any naturally occurring amino acid
XICYNHQSTTRATTKSCEENSCYKKYWRDHRGTIIERGCGCPKVKPGVGIHCCQSDKCNY

SEQ ID NO: 10 - protein, 60 aa, Dendroaspis polylepis
RICYNHQSTTRATTKSCEENSCYKKYWRDHRGTIIERGCGCPKVKPGVGIHCCQSDKCNY

SEQ ID NO: 11 - protein, 72 aa, Dendroaspis polylepis
RTCNKTFSDQSKICPPGENICYTKTWCDAWCSRRGKIVELGCAATCPKVKAGVGIKCCSTDNCNLFKFGKPR

SEQ ID NO: 12 - protein, 72 aa, Dendroaspis polylepis
RTCNKTFSDQSKICPPGENICYTKTWCDAWCSQRGKRVELGCAATCPKVKAGVEIKCCSTDDCDKFQFGKPR

SEQ ID NO: 13 - protein, 91 aa, Ophiophagus hannah
MKTLLLTLVVMTIVCLDLGYTLICFISSHDSVTCAPGENVCFLKSWCDAWCGSRGKKLSFGCAATCPKVNPGIDIECCSTDNCNPHPKLRP

SEQ ID NO: 14 - protein, 72 aa, Ophiophagus hannah
TKCYKTGDRIISEACPPGQDLCYMKTWCDVFCGTRGRVIELGCTATCPTVKPHEQITCCSTDNCDPHHKMLQ

SEQ ID NO: 15 - protein, 72 aa, Ophiophagus hannah
TKCYKTGDISISEACPPGQDLCYMKTWCDVFCGTRGRVIELGCTATCPTVKPHEQITCCSTDNCDPHHKMLQ

SEQ ID NO: 16 - protein, 84 aa, Ophiophagus hannah
MKTLLLTLVVVTIVCLDLGYTRKCLNTPLPLIYKTCPIGQDKCIKMTIKKLPSKYDVIRGCIDICPKSSADVEVLCCDTNKCNK

SEQ ID NO: 17 - protein, 78 aa, Ophiophagus hannah
MKNLLLTFLVVTIVCLDLGYTLICHRVHGLQTCEPDQKFCFRKTTMFFPNHPVLLMGCTYSCPTEKYSVCCSTDKCNK

SEQ ID NO: 18 - protein, 78 aa, Ophiophagus hannah
MKNLLLTFLVVTIVCLDLGYTLICHQVHGLQTCEPAQKFCQIRTTMFFPNHPVLLMGCTYNCPTERYSVCCSTDKCNK

SEQ ID NO: 19 - protein, 86 aa, Ophiophagus hannah
MKTLLLTLVVVTIVCLDLGHTLICVKQYTIFGVTPEICADGQNLCYKTWHMVYPGGYDHTRGCAATCPKMKNHDTVHCCTTDKCNL

SEQ ID NO: 20 - protein, 77 aa, Ophiophagus hannah
MKNLLLTFLVVTIVCLDLGYTLICNRVHGLQTCEPAHKFCFSKTVMPFPNHPLTLMGCTYSCPTERNAVCCSTDKCN

SEQ ID NO: 21 - protein, 84 aa, Ophiophagus hannah
MKTLLLTLVVVTIVCLDLGYTRKCLNTPLPLIYTTCPIGQDKCVKMTIKKLPSKYDVIRGCIDICPKSSADVEVLCCDTNKCNK

SEQ ID NO: 22 - protein, 78 aa, Ophiophagus hannah
MKNLLLTFLVVTIVCLDLGYTLICHQRHGLQTCEPAQKFCFAQTVMPFPNHPLTLMGCTYSCPTEKNAVCCSTDKCNR

(19) United States
(12) Patent Application Publication
(10) Pub. No.: US 2021/0008177 A1
(43) Pub. Date: Jan. 14, 2021

(54) Elapidae neurotoxin enhances opioid analgesic effect and inhibits opioid induced hyperalgesia and tolerance

(71) Applicants: Zhankai Qi, Richmond (CA); Hyatt Qi, Richmond (CA)

(72) Inventors: Zhankai Qi, Richmond (CA); Hyatt Qi, Richmond (CA)

(21) Appl. No.: 16/812,529

(22) Filed: Mar. 9, 2020

(30) Foreign Application Priority Data
Jul. 14, 2019 (CN) 2019106533746

(51) Int. Cl. A61K 38/48; A61P 25/00

(52) U.S. Cl. CPC A61K 38/4806; A61K 45/06; A61P 25/00

(57) ABSTRACT

Provided herein is elapidae neurotoxin, and methods for using a pharmaceutically effective amount of said compound to produce synergistic analgesic effect with an opioid for the treatment of pain. In addition, opioid induced hyperalgesia and tolerance can also be alleviated by said compound while administrated separately, or jointly with the opioid.

### BACKGROUND OF THE INVENTION

### 1. Field of the Invention

The field of the present invention relates to a composition of mater of elapidae neurotoxin, which is a nicotinic acetylcholine receptors (nAChR) modulator (inhibitors or antagonists of nAChR) that can bind to nAChR to produce analgesic and anti-inflammatory effect. Elapidae neurotoxin, while combining with an opioid, they can produce synergistic analgesic effect, and on top of that, opioid induced hyperalgesia and tolerance can also be controlled by elapidea neurotoxin. Pharmaceutical formulations including the elapidae neurotoxin , independently or in combination with an opioid, and a pharmaceutically acceptable carrier base for use in the treatment of aforementioned conditions thereof.

### 2. Description of the Prior Art

Opium is a substance extracted from poppy plants which binds to the opioid receptors of the human body itself and provides pain relief or analgesia to patients with acute or chronic pain. Morphine is an example of an opioid analgesia. Short-term use of opioid drugs is considered as a safe pain relief method. However, when they are frequently administered and/or overused, a series of problems arise which include hyperalgesia, tolerance build-up, drug dependency, constipation, respiratory Inhibition, and fatal overdose. According to the CDC, In 2015, over 33,000 people died in US due to opioid-related overdoses.

Opioid receptor agonists such as morphine are the most widely used potent analgesic drug in clinics. The medicine can effectively relieve the pain of patients, especially post-operative and advanced cancer pain. However, the use of opioids such as morphine often leads to tolerance build-up and hyperalgesia in patients which can occur in as little as a week. And once tolerance and hyperalgesia develops, the patient requires increasing dosages to maintain the drug's initial effectiveness. In other words, the body needs a greater dose of medication to achieve the same analgesic effect as it initially provided; larger doses lead to greater hyperalgesia, the faster build-up of tolerance, constipation, addiction, and respiratory suppression. Therefore, to achieve both the clinical efficacy and the acceptance of treatment, opioid induced hyperalgesia and tolerance are urgent problems in need of a real solution.

Opioid induced analgesic tolerance and hyperalgesia are two closely related yet different symptoms. The former refers to the significant decline in analgesic effects of an opioid, whilst the latter refers to the patient's abnormal pain response to non-injurious stimuli or a highly sensitive pain response to the same injurious stimuli caused by continuous or incorrect applications of opioid receptor agonists such as morphine. Analgesic tolerance and hyperalgesia, although two different adverse reactions induced by opioid receptor agonists, both impede the long-term clinical use of opioids. Consequently, patients under the long-term prescription of such drugs are left to face severe side effects, thus a major unmet clinical need.

Presently, treatments for opioids induced tolerance and hyperalgesia mostly remain in experimental stages. Although the combination of opioids and other drugs has become an effective strategy for enhancing the analgesic effect of opioids, and to reduce the opioid dose, however, there is no singular drug with proven efficacy that could produce synergistic effect, and at same time, address analgesic tolerance and hyperalgesia. Thus clinically, there is a demand for a product that can fully satisfy the unmet needs of patients.

### SUMMARY OF THE INVENTION

The analgesic effect of elapidae neurotoxins have been previously documented (US Patent Application Number 16403651). However, elapidae neurotoxin's ability to inhibit or control opioid induced hyperalgesia and tolerance has never been reported before. Elapidae neurotoxins have no dependence on the opioid system, and no hyperalgesia or tolerance were observed during analgesic process. When combined with opioids, the combination can produce a synergistic analgesic effect and prolong opioid's effective time. These unique properties were also first proved by our invention, thereby the elapidae neurotoxin is expected to be developed into a safe and effective analgesic agent.

The primary purpose of the invention is to provide a composition of matter using elapidae neurotoxin to inhibit or to control the hyperalgesia and tolerance caused by opioids.

The further purpose of the invention is to provide a composition of matter to produce a synergistic analgesic effect for the treatment of pain by combining an elapidae neurotoxin with an opioid, thus avoid to increase the dose of an opioid. Finally, the invention is to provide a composition of matter for treating the patient not responding to opioid (morphine) as an mono therapy, but satisfying with the combination of an opioid and an elapidae neurotoxin.

### BRIEF DESCRIPTION OF THE DRAWINGS

FIG. 1 is the line chart of four days average baseline pain threshold (mechanical tail pressure units (g)) test results of mice randomly divided into 4 groups, namely ‘physiological saline group’, ‘morphine group’, ‘cobrotoxin group’, and ‘cobrotoxin+morphine group’. There is no significant difference between the results of the 4 groups. Cobrotoxin of amino acid sequence ID No.1 will be used for the test.

FIG. 2 is the line chart of four days average baseline pain threshold (mechanical tail pressure units (g)) test results of mice randomly divided into 4 groups, namely ‘physiological saline group’, ‘morphine group’, ‘cobrotoxin group’, and ‘cobrotoxin+morphine group’. There is no significant difference between the results of the 4 groups. Cobrotoxin of amino acid sequence ID No.2 will be used for the test.

FIG. 3 is the average pain threshold curve (mechanical tail pressure units (g)) measured during day 5 through day 11 (total of 7 days) of 4 groups of mice to indicate the hyperalgesia induced by administration of 4 different drugs which were “Physiological saline”, “morphine”, “cobrotoxin”, and “cobrotoxin+morphine” respectively. Cobrotoxin of amino acid sequence ID No.1 was used. The ## symbols indicate a significant statistical difference between the average pain threshold of “morphine group” and the “cobrotoxin+morphine group”, P<0.05. No significant statistical differences were detected between “Physiological saline group”, “cobrotoxin group”, and“cobrotoxin +morphine group”.

FIG. 4 is the average pain threshold curve (mechanical tail pressure units (g)) measured during day 5 through day 11 (total of 7 days) of 4 groups of mice to indicate the hyperalgesia induced by administration of 4 different drugs which were “Physiological saline”, “morphine”, “cobrotoxin”, and “cobrotoxin+morphine” respectively. Cobrotoxin of amino acid sequence ID No.2 was used. The ## symbols indicate a significant statistical difference between the average pain threshold of “morphine group” and the “cobrotoxin +morphine group”, P<0.05. No significant statistical differences were detected between “Physiological saline group”, “cobrotoxin group”, and“cobrotoxin +morphine group”.

FIG. 5 is the average pain threshold column chart (mechanical pressure units (g)) measured during the fifth, eighth, and eleventh day, one hour after injections of 4 different drugs, which were ‘morphine’, ‘physiological saline’, ‘cobrotoxin’, and ‘cobrotoxin+morphine’ respectively. The results of 4 groups of mice reflect the effects of analgesic tolerance. Cobrotoxin amino acid sequence ID No.1 was used. Symbols ### mean a significant statistical difference between the average pain threshold of the “morphine group” and the “cobrotoxin+morphine group” for the day five, day eight, and day eleven, P<0.01; Symbols ### also indicate a significant statistical difference of average pain threshold within the morphine group of day five, day eight, and day eleven, P<0.01.

Symbols *** represent a significant statistical difference of average pain threshold between the “cobrotoxin group” and the “cobrotoxin +morphine group” for the day five, day eight, and day eleven, P<0.01.

FIG. 6 is the average pain threshold column chart (mechanical pressure units (g)) measured during the fifth, eighth, and eleventh day, one hour after injections of 4 different drug , which were ‘morphine’, ‘physiological saline’, ‘cobrotoxin’, and ‘cobrotoxin+morphine’ respectively. The results of 4 groups of mice reflect the effects of analgesic tolerance. Cobrotoxin amino acid sequence ID No.2 was used. Symbols ### mean a significant statistical difference between the average pain threshold of the “morphine group” and the “cobrotoxin+morphine group” for the day five, day eight, and day eleven, P<0.01; Symbols ### also indicate a significant statistical difference of average pain threshold within the morphine group of day five, day eight, and day eleven, P<0.01.

Symbols *** represent a significant statistical difference of average pain threshold between the “cobrotoxin group” and the “cobrotoxin+morphine group” for the day five, day eight, and day eleven, P<0.01.

FIG. 7 is the column charts of rat writhing numbers counted at time intervals of 60 minutes, 150 minutes, and 210 minutes after injecting 1 ml of 1.5% acetic acid solution in SD rats at each time interval. Cobrotoxin of amino acid sequence ID No.1 was used.

Symbols ### show a significant statistical difference in the number of writhing between “morphine group” and “cobrotoxin+morphine group” at 60, 150, and 210 minutes time intervals after injection, P<0.01; Symbols ### also indicate a significant statistical difference in the number of writhing within the morphine group between 60, 150, and 210 minutes time intervals after injection, P<0.01.
Symbols *** indicate significant statistical differences in the number of writhing between “cobrotoxin group” and “cobrotoxin +morphine group” at 60, 150, and 210 minutes time intervals after injection, P<0.01.

FIG. 8 is the column charts of rat writhing numbers counted at time intervals of 60 minutes, 150 minutes, and 210 minutes after injecting 1 ml of 1.5% acetic acid solution in SD rats at each time interval. Cobrotoxin of amino acid sequence ID No.2 was used.

Symbols ### show a significant statistical difference in the number of writhing between “morphine group” and “cobrotoxin+morphine group” at 60, 150, and 210 minutes time intervals after injection, P<0.01; Symbols ### also indicate a significant statistical difference in the number of writhing within the morphine group between 60, 150, and 210 minutes time intervals after injection, P<0.01.
Symbols ** and *** indicate significant statistical differences in the number of writhing between “cobrotoxin group” and “cobrotoxin +morphine group” at 60, 150, and 210 minutes time intervals after injection, P<0.05 and P<0.01 respectively.

### DETAILED DESCRIPTION OF THE INVENTION

Most widespread of the snake venom neurotoxins are the post synaptically active alpha neurotoxins (αNtx), and they are found widely in Elapidae and Hydrophiid venoms [J. White et al, 1996].

Elapidae neurotoxins are antagonists of nicotinic acetylcholine receptors (nAChR) which bind to muscle and neuronal nAChR in an antagonistic and slow reversible manner. Such elapidae neurotoxins are known as postsynaptic neurotoxins or alpha-neurotoxins due to their ability to block nAChR [Naguib M et al, 2002; Abbas M et al, 2016]. Structurally they have a three-finger appearance, with the active site near the tip of the middle finger [J. White et al, 1996], and this three-finger appearance is a multifunctional structural scaffold able to modulate cholinergic functions [Pascale Marchot et al, 2017].

nAChR influences pain, senses, cognition, neuronal protection, and neurotransmitter transmission [Li Jiangbing et al, 2017]. Elapidae neurotoxins produce analgesic effects through modulating nAChR without the involvement of the opioid receptors system. When combine with an opioid, elapidae neurotoxins can synergize the analgesic effect through anti-inflammatory function.

According to published experimental data, pro-inflammatory cytokines are associated with various types of pain, one of which is pathological neuralgia. Neuropathic pain, pain caused by artificial subcutaneous formalin injection or subarachnoid injection increases the secretion of IL-1B level significantly, whilst blocking IL-1B receptors can reduce pain [Milligan et al, 2001]. IL-6 can induce mechanical pain sensitivity and hyperalgesia, knockout IL-6 gene can inhibit pain in rats with sciatic nerve ligation [Murphy et al, 1999]. Pro-inflammatory cytokines can increase pain in several ways, in the presence of a cytokine receptor on the neurons, pro-inflammatory cytokines may act directly on the neurons of the central nervous system to augment pain; pro-inflammatory cytokines can augment pain by modulating the transmission of incoming neural signals onto primary nerve fibers as well.

Pro-Inflammatory cytokines can also induce astrocytes and small glial cells to increase the synthesis and release of nitric oxide (NO) and activate nitric oxide synthase (NOS). These substances indirectly increase the magnitude of pain [Xiang hongbing et al, 2004; Haberberger et al, 2003; Rainer Viktor et al, 2002; Papadopolou. S et al, 2004; Watkins et al, 2001]. According to published experimental data, morphine-induced hyperalgesia and tolerance are accompanied by high levels of IL-1, IL-6, NOS activity, and NO content [liang huichun, 2014; Jian daolin 2005]. Experimental data also show that numerous nicotinic acetylcholine receptor (nAChR) acts as an important intermediate link in regulating pro-inflammatory cytokines, NOS activity, and NO content. nAChR antagonists either directly reduce pro-inflammatory cytokines, NOS activity or NO content, or activate certain specific nAChR (e.g., a7-nAChR, a9-nAChR), to reduce pro-inflammatory cytokines, NOS activity or NO content [Zakrzewicz A, J et al, 2017; Patel et al, 2017; Papadopolou S, et al, 2004; Thippeswamy T, et al, 2001; Richter K, et al, 2016]. Other experimental results demonstrate that nAChR antagonists are directly involved in the process of reducing neuropathic pain [Pacini A et al, 2016; Romero H K et al, 2017; Vincler M et al, 2006; Luo S, et al, 2015; Holtman J R et al, 2011; Wala E P et al, 2012].

Elapidae neurotoxin, as the major antagonist of nicotinic acetylcholine receptor, has been shown in our experiments to be able to reduce pro-inflammatory cytokines, NOS activity and NO content, which is in line with the reported function of other nicotinic acetylcholine receptor antagonists.

Elapidae neurotoxins, on top of its independent analgesic effect, exhibit strong anti-inflammatory properties as well, and patients under opioids induced hyperalgesia and tolerance experience neuron inflammation, therefore, elapidae neurotoxins demonstrate dual mechanisms while treating opioids induced hyperalgesia and tolerance.

The main elapidae neurotoxins include cobrotoxins, bungarotoxins, neurotoxins from black mamba, and neurotoxins from king cobra, they all have the common three-finger appearance structure. The following elapidea neurotoxins were proved effective in enhancing opioid analgesic effect and in inhibiting opioids induced hyperalgesia and tolerance in our experiments.

Cobrotoxin of amino acid
sequence ID No. 1
(lechnqqssq tptttgcsgg etncykkrwr dhrgyrterg
cgcpsvkngi einccttdrc nn)
Cobrotoxin of amino acid
sequence ID No.2
(mktllltllv vtivcldlgy tlechnqqss qtptttgcsg
getncykkrw rdhrgyrter gcgcpsvkng ieinccttdr cnn)
Cobrotoxin of amino acid
sequence ID No. 3
(lechnqqssq tptttgcsgg etncykkrwr dhrgyrterg
cgcpivkngi esnccttdrc nn)
Cobrotoxin of amino acid
sequence ID No. 4
(mechnqqssq apttktcsge tncykkwwsd hrgtiiergc
gcpkvkpgvn lnccttdrcnn)
Cobrotoxin of amino acid
sequence ID No. 5
(mechnqqssq tptttgcsgg etncykkwws dhrgtiierg
cgcpkvkpgv nlnccttdrcnn)
Cobrotoxin of amino acid
sequence ID No. 6
(lechnqqssq tpttktcsge tncykkwwsd hrgtiiergc
gcpkvkpgvn lnccttdrcnn)
Bungarotoxins of amino acid
sequence ID No. 7
(ivchttatsp isavtcppge nlcyrkmwcd afcssrgkvv
elgcaatcps kkpyeevtcc stdkcnphpk qrpg)
Bungarotoxin of amino acid
sequence ID No. 8
(mktllltlvv vtivcldlgy tivchttats pisavtcppg
enlcyrkmwc dafcssrgkv velgcaatcp skkpyeevtc
cstdkcnphp kqrpg)
Black Mamba Neurotoxin of amino acid
sequence ID No. 9
(xicynhqstt rattksceen scykkywrdh rgtiiergcg
cpkvkpgvgi hccqsdkcny)
Black Mamba Neurotoxin of amino acid
sequence ID No. 10
(ricynhqstt rattksceen scykkywrdh rgtiiergcg
cpkykpgvgi hccqsdkcny)
Black Mamba Neurotoxin of amino acid
sequence ID No. 11
(rtcnktfsdq skicppgeni cytktwcdaw csrrgkivel
gcaatcpkvk agvgikccst dncnlfkfgk pr)
Black Mamba Neurotoxin of amino acid
sequence ID No. 12
(rtcnktfsdq skicppgeni cytktwcdaw csqrgkrvel
gcaatcpkvk agveikccst ddcdkfqfgk pr)
King cobra neurotoxins of amino acid
sequence ID No. 13
(mktllltlvv mtivcldlgy tlicfisshd svtcapgenv
cflkswcdaw cgsrgkklsf gcaatcpkvn pgidieccst
dncnphpklr p)
King cobra neurotoxins of amino acid
sequence ID No. 14
(tkcyktgdri iseacppgqd lcymktwcdv fcgtrgrvie
lgctatcptv kpheqitccs tdncdphhkm lq)
King cobra neurotoxins of amino acid
sequence ID No. 15
(tkcyktgdri iseacppgqd lcymktwcdv fcgtrgrvie
lgctatcptv kpheqitccs tdncnphpkm kq)
King cobra neurotoxins of amino acid
sequence ID No. 16
(mktllltlvv vtivcldlgy trkclntplp liyktcpigq
dkcikmtikk lpskydvirg cidicpkssa dvevlccdtn kcnk)
King cobra neurotoxins of amino acid
sequence ID No. 17
(mknllltflv vtivcldlgy tlichrvhgl qtcepdqkfc
frkttmffpn hpvllmgcty scptekysvc cstdkcnk)
King cobra neurotoxins of amino acid
sequence ID No. 18
(mknllltflv vtivcldlgy tlichqvhgl qtcepaqkfc
qirttmffpn hpvllmgcty ncpterysvc cstdkcnk)
King cobra neurotoxins of amino acid
sequence ID No. 19
(mktllltlvv vtivcldlgh tlicvkqyti fgvtpeicad
gqnlcyktwh mvypggydht rgcaatcpkm knhdtvhcct tdkcnl)
King cobra neurotoxins of amino acid
sequence ID No. 20
(mknllltflv vtivcldlgy tlicnrvhgl qtcepahkfc
fsktvmpfpn hpltlmgcty scpternavc cstdkcn)
King cobra neurotoxins of amino acid
sequence ID No. 21
(mktllltlvv vtivcldlgy trkclntplp liyttcpigq
dkcvkmtikk lpskydvirg cidicpkssa dvevlccdtn kcnk)
King cobra neurotoxins of amino acid
sequence ID No. 22
(mknllltflv vtivcldlgy tlichqrhgl qtcepaqkfc
faqtvmpfpn hpltlmgcty scpteknavc cstdkcnr)

The amino acid sequences of the above elapidea neurotoxins are submitted separately in ASCII text file in the name of “sequence listing”, created 2020-Aug.-12, with size of 16 KB.

The following examples are provided to illustrate, but not limit the invention.

### EXAMPLES

### Example A

Elapidae neurotoxin preparation

Separation and Purification of cobrotoxin of amino acid sequence ID No. 1

Based on lyophilized venom powder from Naja atra, a total of 12 fractions were isolated by cation-exchange chromatography on an open column (50×2.5 cm I.D.) packed with TSK CM-650(M). The process was performed and described in the following sequence:
i. Venom powder was dissolved in 10 ml of 0.025 M ammonium acetate (pH6.0).
ii. Starting buffer (20-50 mg/ml) was applied to TSK CM-650 column equilibrated with the same buffer.
iii. After the column had been washed with 300 ml of the initial buffer, the proteins adsorbed were eluted with a two-stage linear gradient (0.1-0.5 M and 0.7-1.0 M ammonium acetate buffer).
iv. A reverse-phase HPLC (RP-HPLC) was performed on a Hitachi' liquid chromatograph with a model L-6200 pump. The column eluates (6 ml/tube/7.5 min) were monitored for absorbance at 280 nm.
v. A total of 12 fractions from the aforementioned ion-exchange chromatography were further desalted and purified by a reverse-phase HPLC (RP-HPLC) with Vydac RP-C18 (4.6×250 mm, 5.0 um).
vi. The amino acid sequences of 12 fractions were further analyzed using Edman degradation method.
vii. Cobrotoxin of amino acid sequence ID No. 1 was identified.

### Example B

### In Vivo Anti-Hyperalgesia/Tolerance Model

To evaluate the therapeutic effects elapidea neurotoxins, one of the reliable morphine induced hyperalgesia/tolerance model in mice (Elhabazi, K et al) was created, and effects of the representative EXAMPLE compounds were investigated on the model.

### Morphine-Induced Hyperalgesia and Analgesic Tolerance Model

| | Mice of morphine group receiving morphine injection from day | | | | | | |
|---|---|---|---|---|---|---|---|
| | 5-day 11 for 7 consecutive days to induce hyperalgesia and tolerance | | | | | | |
| Mice average pain threshold | Day 5 | Day 6 | Day 7 | Day 8 | Day 9 | Day 10 | Day 11 |

| baseline measurement for 4 days | | | | Measurement of pain threshold before injections from day 5 to day 11 |
|---|---|---|---|---|
| Day 1 | Day 2 | Day 3 | Day 4 | for 7 consecutive days to test the morphine-induced hyperalgesia |

| | | |
|---|---|---|
| Measuring | Measuring | Measuring |
| tolerance | tolerance | tolerance |
| after | after | after |
| injection | injection | injection |
| in day 5 | in day 8 | in day 11 |

Cobrotoxin of amino acid sequence ID No.1 and cobrotoxin of amino acid sequence ID No.2 were used in parallel for the tests.

Detailed steps are as follows:

Step1. Establishment of mice's baseline pain threshold.

100 Kunming mice were subjected to tail pressure tests for 4 days to measure the mechanical pain threshold and the average pain threshold will be set as the baseline pain threshold.
i. Put the mouse gently into the restraint and put its tail under the conical tip of the pain test apparatus. Press the pedal switch, and increase the pressure on the proximal end of the tail evenly until signs of the first pain response (struggle, squeaking) occurs. Record pain-inducing pressure when pain response occurs (units: g) as the value of pain threshold. The pressure was released upon reaching 700 g with no indication of a response to avoid tissue injury. Measurements were also conducted at the middle and distal ends of the tail of the same mouse with a minimum of 30 seconds time intervals between each measurement.
ii. Put the tested mice in a cage and test the next one until every mouse in the group is tested. The average of the three measurements (i.e., the proximal, mid and distal ends of each mouse tail) was used as the pain threshold (g) of each mouse when all mice were subjected to a tail pressure test.
iii. In the following 3 days, all mice underwent repeated measurements of tail pressure pain threshold.
iv. The pain threshold of the mice measured by the tail pressure test ranged from 180 g to 220 g. The mice were then randomly divided into four groups: namely ‘physiological saline group’, ‘morphine group’, ‘cobrotoxin group’ and ‘cobrotoxin+morphine group’. Each group comprised of 20 mice, which is used for the hyperalgesia and tolerance test of the corresponding drugs in the group's name. Any surplus mice were excluded.
v. Lastly, each group of 20 mice was divided randomly again into two groups, with each group comprising 10 mice, as cobrotoxin of amino acid sequence ID NO.1, and cobrotoxin of amino acid sequence ID NO. 2 will be used for parallel testing

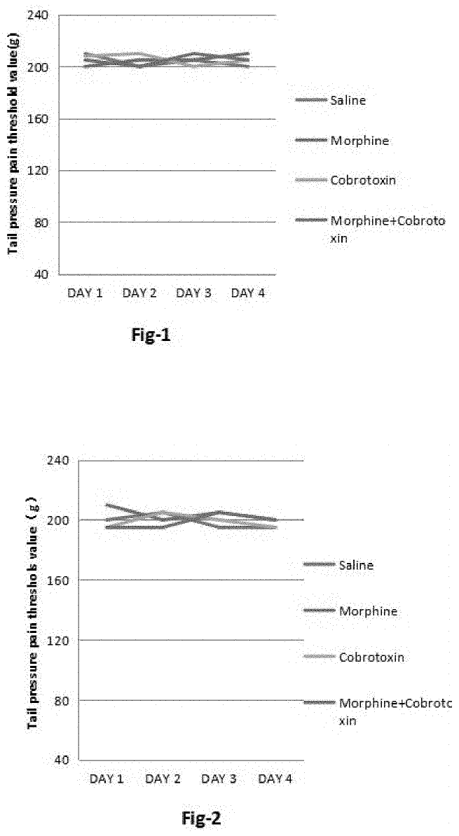

As we can see there were no significant statistical differences between the 4 groups of mice in both FIG. 1 and FIG. 2.

Step2. Morphine-Induced hyperalgesia and analgesic tolerance in mice

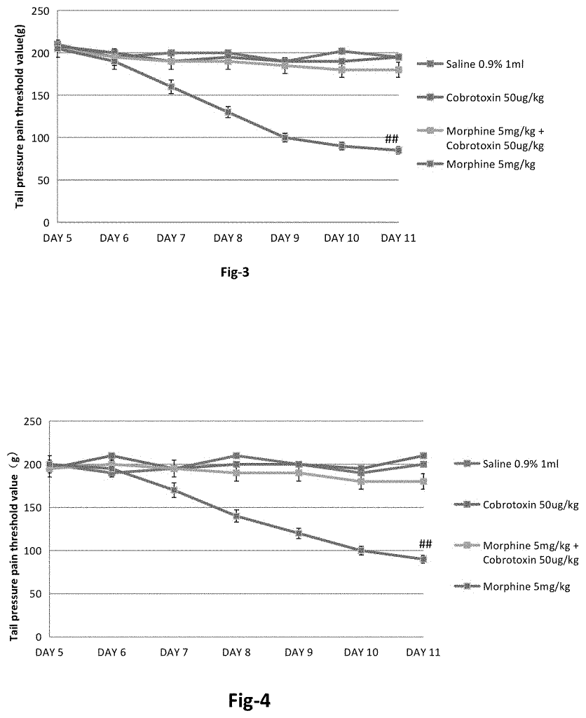

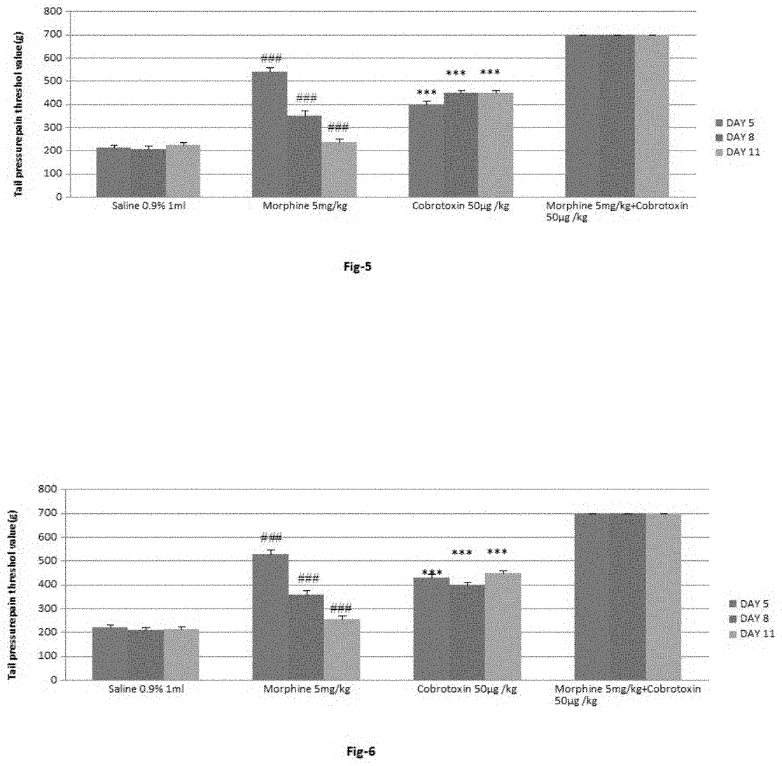

After 4 days of measurement of the mice's baseline pain threshold described in step 1, from the fifth day to the eleventh day, the measurement of pain threshold was performed before the injection. Each 4 groups of mice (total 8 groups) underwent the tail pressure test first and then were injected with morphine (5 mg/kg), sterile saline (NaCl 0.9% 1 ml), cobrotoxin (50 ug/kg), or cobrotoxin (50 ug/kg)+morphine (5 mg/kg) respectively. The test results indicate that the mean pain threshold of the “morphine group” is significantly lower than that of the other 3 controlled groups. Cobrotoxin of amino acid sequence ID NO.1, and cobrotoxin of amino acid sequence ID NO. 2 were used for parallel testing. The experimental data is shown in FIG. 3 and FIG. 4.

Parallelly, in the 5th, 8th, and 11th day, an hour after the injection of 4 different drugs, the tail pressure test was applied again to measure the pain threshold of each mouse of the “morphine group”, the “physiological saline group”, the “cobrotoxin group”, and the “cobrotoxin +morphine group” to determine the analgesic tolerance of these four drugs.

The test results indicate that within the “morphine group”, the mean pain threshold was significantly decreased from day 5 to day 11, with highest in day 5, and lowest in day 11; between the “morphine group” and “morphine+cobrotoxin group”, the mean pain threshold was also different. The mean pain threshold of the ‘cobrotoxin+morphine group’ is higher than that of ‘cobrotoxin group’, and the difference was statistically significant for day 5, 8, and 11 as well. The experimental data suggests that the cobrotoxin can inhibit morphine-induced analgesic tolerance, and cobrotoxin+morphine can produce a stronger analgesic effect than morphine or cobrotoxin alone.

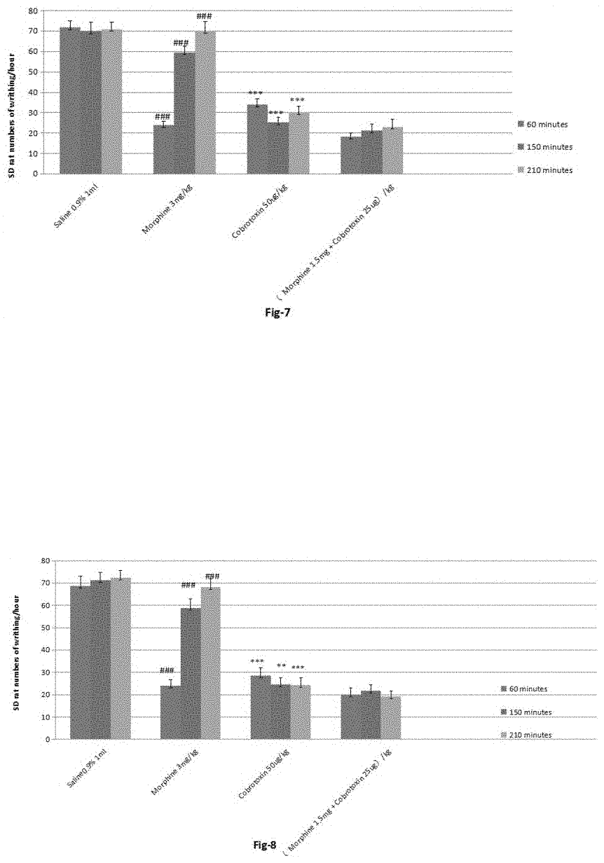

Cobrotoxin of amino acid sequence ID NO.1, and cobrotoxin of amino acid sequence ID NO. 2 were used for parallel testing. The experimental data is shown in FIG. 5 and FIG. 6.

### Example C

### In Vivo Synergistic Analgesic Effects and Prolongation of Morphine's Analgesia Time Model

Synergistic analgesic effect and ability to prolong morphine's effective time of the representative EXAMPLE compounds were investigated on the model. Writhing test was applied to the rats, which is a chemical method used to induce pain of the peripheral origin by injection of irritant principles like acetic acid in rats. Analgesic effect of the test compound is inferred from the decrease in the frequency of writhe.

Four groups of rats (10/group) were injected with sterile saline
(NaCl 0.9% 1 ml), morphine (3 mg/kg), cobrotoxin (50 ug/kg),
or cobrotoxin (25 ug/kg) + morphine (1.5 mg/kg) respectively.

| 60 minutes after | 150 minutes after | 210 minutes after |
|---|---|---|
| the initial | the initial | the initial |
| injection | injection | injection |
| Injection of 1.5% | Injection of 1.5% | Injection of 1.5% |
| acetic acid solution | acetic acid solution | acetic acid solution |
| for writhing test | for writhing test | for writhing test |
| To test the | To test cobrotoxin's | To test cobrotoxin's |
| synergistic analgesic | ability to prolong | ability to prolong |
| effect of cobrotoxin | morphine's | morphine's analgesia |
| combined with morphine | analgesia time | time |

Details as follows:

Step1. Synergistic analgesic effect of cobrotoxin combined with morphine 80 SD rats were randomly divided into “physiological saline group”, “morphine group”, “cobrotoxin group” and “cobrotoxin+morphine group” with 20 rats in each group, and finally, each group will be divided again into two groups for 2 cobrotoxins parallel testing.

The aforementioned four groups of rats were injected with sterile saline (NaCl 0.9% 1 ml), morphine (3 mg/kg), cobrotoxin (50 ug/kg), and cobrotoxin (25 ug/kg)+morphine (1.5 mg/kg) respectively.

60 minutes after injection, 1.5% acetic acid solution was injected to SD rats (1 ml/rat). experiment results show the analgesic effect provided by half dose cobrotoxin (25ug/kg)+half dose morphine (1.5 mg/kg) is significantly higher compared to a single full dose of morphine (3 mg/kg), or a single full dose of cobrotoxin (50 ug/kg). This means that the cobrotoxin+morphine produces superior analgesic improvement rather than an additive one, indicating a synergistic analgesic effect.

Cobrotoxin of amino acid sequence ID NO.1, and cobrotoxin of amino acid sequence ID NO. 2 were used for parallel testing. The experimental data is shown in FIG. 7 and FIG. 8

Step2. Prolongation of morphine's analgesia effect by cobrotoxin

Following Step1, the aforementioned four groups SD rats were injected with 1.5% acetic acid solution (1 ml/rat) again 150 and 210 minutes after the initial injection of 4 different drugs respectively.

The test result indicated that SD rats of morphine group showed lower analgesic effect after 150 minutes, and almost no signs of any analgesic effect after 210 minutes; the SD rats of cobrotoxin group retained signs of analgesic effect but was inferior in comparison with the SD rats of cobrotoxin+morphine group with a significant statistical difference. The test results demonstrate the synergy formed when combining half a dose of cobrotoxin and half a dose of morphine. The combination has a stronger analgesic effect than a single full dose of cobrotoxin or morphine, and this synergistic effect did not decline with the decrease of morphine's analgesic effect at 150 and 210 minutes, which showed a prolonged analgesic effect of morphine through combination with cobrotoxin.

After the four groups of SD rats received injection of their respective drugs, the mean number of writhing per hour measured after 60, 150, and 210 minutes of initial injection was shown in FIG. 7 (cobrotoxin of amino acid sequence ID NO.1 was used), and FIG. 8 (cobrotoxin of amino acid sequence ID NO.2 was used).

### Example D

Test of pro-inflammatory cytokines IL-113 and IL-6, NOS activity, and NO content in tissues of mice.

Further studies were conducted on the mechanism of cobrotoxin's inhibition of hyperalgesia and tolerance, which were mainly focused on the determination of IL-1β and IL-6 blood level, NOS activity, and NO content at tissues of mice.

The specific steps were as follows:
i. After completion of the aforementioned morphine tolerance and hyperalgesia tests, mice of “morphine group” and “cobrotoxin +morphine group” were set aside for 2 hours followed by anesthesia with chloral hydrate, and then dislocated.
ii. The extraction of the lumbar spinal cord was performed quickly on a plate with ice, then washed with icy water.
iii. The spinal tissues, weighed, then put into pre-frozen physiological saline, 4000 revolution/separation for 10 minutes, and prepared into 10% homogenate which was measured for IL-1β, IL-6, NOS activity, and NO content.
iv. The ELISA method was applied to determine the tested values of the lumbar spinal cord tissues. The release of IL-1β, IL-6, NOS and NO was detected according to the instruction in the insert.
v. Coomassie brilliant blue dye was used to determine the total protein content in the homogenate of each sample.

The levels of IL-1β, IL-6, NOS activity, and NO content detected in the “morphine group” and “cobrotoxin+morphine group” were as follows: (cobrotoxin of amino acid sequence ID NO.1 was used for the test)

| Biomarker | Group | Value | SD | t-test |
|---|---|---|---|---|
| IL-1β pg/mg protein | Morphine Group | 16.80 | 2.39 | P < 0.01 |
| | Morphine + cobrotoxin | 11.30 | 1.83 | |
| | Group | | | |
| IL-6 pg/mg protein | Morphine Group | 19.40 | 2.12 | P < 0.01 |
| | Morphine + cobrotoxin | 14.80 | 1.75 | |
| | Group | | | |
| NOS U/mg protein | Morphine Group | 7.56 | 0.14 | P < 0.01 |
| | Morphine + cobrotoxin | 7.15 | 0.12 | |
| | Group | | | |
| NO μmol/g protein | Morphine Group | 1.61 | 0.03 | P < 0.01 |
| | Morphine + cobrotoxin | 1.32 | 0.02 | |
| | Group | | | |

The experimental data showed that the level of IL-1B, IL-6, NOS activity, and NO content of the “morphine group” were significantly higher than that of “cobrotoxin+morphine group”.

Although the invention herein has been described with reference to particular embodiments, it is to be understood that these embodiments are merely illustrative of the principles and applications of the present invention. It is, therefore, to be understood that numerous modifications may be made to the illustrative embodiments and that other arrangements may be devised without departing from the spirit and scope of the present invention as defined by the appended claims.

### REFERENCES

Abbas M, Rahman S. Effects of αα-7 nicotinic acetylcholine receptor positive allosteric modulator on lipopolysaccharide-induced neuroinflammatory pain in mice. Eur J Pharmacol. 783: 85-91, 2016.

Elhabazi, K et al. Assessment of Morphine-induced Hyperalgesia and Analgesic Tolerance in Mice Using Thermal and Mechanical Nociceptive Modalities. J. Vis. Exp. (89), e51264, doi:10.3791/51264, 2014.

Haberberger et al. Nicotinic receptor alpha7-subunits are coupled to the stimulation of nitric oxide synthase in rat dorsal root ganglion neurons. Histochem Cell Biol. 120:173-181, 2003.

Holtman J R et al. The novel small molecule alpha9alpha10 nicotinic acetylcholine receptor antagonist ZZ-204G is analgesic. Eur J Pharmacol 2011.670, 500-508.

J. White et al, 1996 Snake Neurotoxin. Human Toxicology, 1996.

Jian Daolin et al. Effect of morphine tolerance on IL-1B and IL-6 levels in rat spinal cord, Journal of Practical Medicine. Vol. 21, No. 20, 2005.

Khadija Elhabazi et al. Assessment of Morphine-induced Hyperalgesia and Analgesic Tolerance in Mice Using Thermal and Mechanical Nociceptive Modalities

Li Jiangbing et al. Research progress on the interaction between alpha neurotoxin and nicotinic acetylcholine receptors, Life Science, Vol. 29, No. 1, January 2017.

Liang Huichun. Evaluation of Guiyuan's anti-morphine analgesic tolerance and hyperalgesia effect and its mechanism. Chinese Academy of Military Sciences, 2014.

Luo S, et al. Cloning, synthesis, and characterization of alpha conotoxin GeXIVA, a potent alpha9alpha10 nicotinic acetylcholine receptor antagonist. Proc Natl Acad Sci USA. 112, E4026-E4035, 2015.

Milligan et al. Intrathecal HIV-1 envelop glycoprotein gp 120 enhanced states mediated by spinal code proinflammatory cytokines. J neuroscience. (8)2808-2819, 2001.

Murphy et al. Endogenous interleukin-6 contributes to hypersensitivity to cutaneous stimuli and changes in neuropeptides associated with chronic nerve constriction in mice. Eur J Neurosci. 11 (7),2243-2253, 1999.

Naguib M et al. Advances in neurobiology of the neuromuscular junction: implications for the anesthesiologist. J Am Soc Anesthesiol. 96:202-31, 2002.

Pacini A et al. The alpha9alpha10 nicotinic receptor antagonist alpha-conotoxin RgIA prevents neuropathic pain induced by oxaliplatin treatment. Exp Neurol. 282, 37-48, 2016.

Pascale Marchot et al, The three-finger toxin fold: a multifunctional structural scaffold able to modulate cholinergic functions Journal of neurochemistry, Vol 142, issue S2, 2017.

Papadopolou S, et al. Nicotinic receptor mediated stimulation of NO-generation in neurons of rat thoracic dorsal root ganglia. Neurosci Lett 361, 32-35. 77, 2004.

Patel et al. Anti-inflammatory effects of astroglial α7 nicotinic acetylcholine receptors are mediated by inhibition of the NF-κB pathway and activation of the Nrf2 pathway Journal of Neuroinflammation. 14:192, 2017.

Rainer Viktor et al. The role of spinal neuroimmune activation in morphine induced tolerance/hyperplasia in neuropathic and sham operated rats. J Neurosci. 22(22)9980-9989, 2002.

Richter K, et al. Phosphocholine—an agonist of metabotropic but not of ionotropic functions of alpha9-containing nicotinic acetylcholine receptors. Sci Rep. 6, 28660, 2016.

Romero H K et al. Inhibition of a9a10 nicotinic acetylcholine receptors prevents chemotherapy-induced neuropathic pain. Proc Natl Acad Sci USA. 114, 1825-1832, 2017.

Thippeswamy T, et al. Inhibition of neuronal nitric oxide synthase results in neurodegenerative changes in the axotomized dorsal root ganglion neurons: evidence for a neuroprotective role of nitric oxide in vivo. Neuroscience Research. 05, 2001.

Vincler M et al. Molecular mechanism for analgesia involving specific antagonism of alpha9alpha10 nicotinic acetylcholine receptors. Proc Natl Acad Sci USA. 103,17880-17884, 2006.

Wala E P et al. Novel small molecule alpha9alpha10 nicotinic receptor antagonist prevents and reverses chemotherapy-evoked neuropathic pain in rats. Anesth Analg. 115, 713-720, 2012.

Watkins et al. Spinal cord glia new player in pain. Pain. 93(3):201-205, 2001.

Xiang Hongbing et al. Effect of ketamine on astrocytes in spinal cord of morphine tolerant mice. Chinese Journal of Anesthesiology. 24 (4) 290-293, 2004.

Zakrzewicz A, J et al. Canonical and novel noncanonical cholinergic agonists inhibit ATP-induced release of monocytic interleukin-1beta via different combinations of nicotinic acetylcholine receptor subunits alpha7, alpha9 and alpha10. Front Cell Neurosci 11, 189, 2017.

1. Claims:

2. A method for treating opioids induced hyperalgesia in a mammal. Said method comprising administering to a mammal in need thereof a pharmaceutical composition of a therapeutically effective amount of elapidae neurotoxin, and a pharmaceutically acceptable carrier base for use in inhibiting or controlling opioids induced hyperalgesia.

3. A method for treating opioids induced tolerance in a mammal. Said method comprising administering to a mammal in need thereof a pharmaceutical composition of a therapeutically effective amount of elapidae neurotoxin, and a pharmaceutically acceptable carrier base for use in inhibiting or controlling opioids induced tolerance.

4. A method for treating pain in a mammal. Said method comprising administering to a mammal in need thereof a pharmaceutical composition of a therapeutically effective amount of elapidae neurotoxin, and a therapeutically effective amount of opioid, and a pharmaceutically acceptable carrier base for use in producing synergistic or better analgesic effect for the patients not satisfying with an opioid as analgesia.

5. A method for treating pain in a mammal. Said method comprising administering to a mammal in need thereof a pharmaceutical composition of a therapeutically effective amount of elapidae neurotoxin, and a therapeutically effective amount of opioid, and a pharmaceutically acceptable carrier base for use in prolonging the analgesic effect of an opioid while treating pain .

6. A method for treating pain in a mammal. Said method comprising administering to a mammal in need thereof a pharmaceutical composition of a therapeutically effective amount of elapidae neurotoxin, and a therapeutically effective amount of opioid, and a pharmaceutically acceptable carrier base for use in controlling or alleviating the pain in patients who do not respond to an opioid mono therapy .

7. The elapidae neurotoxin according to claim (1-5), characterized in that it is a elapidae neurotoxin polypeptide having the amino acid sequence shown in SEQ ID No. 1 to SEQ ID No. 22; or elapidae neurotoxin polypeptide homologues having 70% or more homology with the elapidae neurotoxin polypeptide of SEQ ID No. 1 to SEQ ID No. 22, and the biological function of the elapidae neurotoxin polypeptide homologues is the same as or similar to that of the elapidae neurotoxin polypeptide of the amino acid sequence ID No. 1 to SEQ ID No. 22.

8. Elapidae neurotoxin polypeptides or elapidae neurotoxin polypeptides homologues according to claim (1-6), characterized in that they can be derived from natural snake venoms, or synthesized from chemical polypeptides, or can be obtained from prokaryotic or eukaryotic hosts using recombinant technology (for example, Bacteria, yeast, higher plants, insects and mammalian cells).

9. The recombinantly produced elapidae neurotoxin polypeptide or its homologues according to claim (7), based on the host used in the recombinant production scheme, the polypeptide or its homologues of the present invention may be glycosylated, or may be non-glycosylated;
Disulfide-bonded or non-disulfide-bonded. The polypeptides and its homologues described in the present invention may also include or exclude the starting methionine residue.

10. The elapidae neurotoxin polypeptide according to claim (1-8), further characterized in that the polypeptide in the present invention may include fragments of the above-mentioned various elapidae neurotoxin polypeptides after hydrolysis or enzymolysis, derivatives or analogs treated by physical, chemical or biological method, they are polypeptides which basically maintain the same biological function or activity as the above-mentioned elapidae neurotoxin polypeptide. The fragments, derivatives or analogs described in the present invention may be a polypeptide in which one or more amino acid residues are substituted, or a polypeptide having a substituent group in one or more amino acid residues, or combined with a compound (such as compounds that extend the half-life of a polypeptide, such as polyethylene glycol), or a polypeptide formed by fusion of a fatty chain, or a polypeptide formed by fusing an additional amino acid sequence to this polypeptide sequence. As described herein, these fragments, derivatives, and analogs are within the scope of those skilled in the art.

11. The method as described in claim (1-5), wherein the respective compounds are administered simultaneously, separately or sequentially.

12. The method as described in claims 3-5, wherein the pain is acute or chronic pain, including traumatic pain, somatic pain, visceral pain, neuropathic pain, post-operative pain, cancer pain, inflammatory pain, fibromyalgia, toothache , Dysmenorrhea, kidney pain, headache, biliary colic, arthralgia, back pain, arthroscopic pain, gynecological laparoscopic pain, and pain caused by burns, rheumatoid arthritis, intraocular hypertension, and virus infection etc.

13. The method as described in claims 1-5 comprising intravenous, intramuscular, subcutaneous, intra-articular, oral, sublingual, nasal, rectal, topical, intradermal, intraperitoneal, intrathecal administration or transdermal administration.

14. The dose of elapidae neurotoxin of the method of claims 1-5 includes from 1 μg/Kg to 350 μg/kg each time, and the injection frequency ranges from once a day to multiple times a day, or multiple times a year.
